(19) United States
(12) Patent Application Publication
(10) Pub. No.: US 2011/0082395 A1
(43) Pub. Date: Apr. 7, 2011

(54) MEDICAL THERAPY DEVICE

(75) Inventors: Michael BURKHARDT, Muehlacker (DE); Francesco MESSINA, Kraichtal (DE); Jean-Georges ZIMMERMANN, Knittlingen (DE)

(73) Assignee: RICHARD WOLF GMBH, Knittlingen (DE)

(21) Appl. No.: 12/897,923

(22) Filed: Oct. 5, 2010

(30) Foreign Application Priority Data
Oct. 6, 2009 (DE) 10 2009 048 361.6

(51) Int. Cl. A61B 17/225

(52) U.S. Cl. 601/2

(57) ABSTRACT

The medical therapy device includes at least one picture-providing locating device, a treatment device, a patient rest, means for the motorized positioning of at least the treatment device relative to the patient rest, means for control of the therapy device, and a display and operating device. The treatment center of the treatment device may be represented on the display in at least one picture of the locating device on the display, and operating elements are provided, with which the positioning in coordinates of the locating device may be controlled.

### BACKGROUND OF THE INVENTION

The invention relates to a medical therapy device, in particular to a sound wave treatment device, as well as to a display and operating device for such a device.

Such devices are part of the prior art and typically consist of a picture-providing locating device, a treatment device, in particular a focusing sound wave treatment device, as well as a patient support, which may be displaced in a motorized manner, so that the location in the patient body, which is to be treated, e.g. a concrement, may be brought to correspond with the treatment center of the treatment device, thus with the focus of the sound wave treatment device. Moreover, means for controlling the therapy device, and a display and operating unit are provided. The control thereby serves on the one hand for activating the treatment device and on the other hand for positioning the patient support. For this, a display is provided, which represents a picture of the locating device as well as furthermore the focus of the sound wave treatment device. Typically, on the one hand an ultrasound locating device and on the other hand an X-ray locating device, are provided as locating devices, wherein a separate display is provided for each of the locating devices. Such a device is known for example from U.S. published patent application 2008/0146908 A1.

The disadvantage with such a treatment device is the fact that an orientation on the display of the locating device to the actual position of the patient or the target in the patient to be treated is often difficult, since the display is located at a certain location in a stationary manner next to the patient support which is typically designed as a rest. The patient support may thereby be displaced in table coordinates as is common with such tables, up-down, left-right and forwards-backwards. These directions, however, do not typically agree with those in the display representation of the locating systems, which is why the therapist, by trial and error, must first find out by which way of table displacement, one may move the focus of the sound wave treatment device in the direction of the target point in the patient. This may lead to the target point, thus for example the concrement to be treated, being lost by displacing the patient support in the wrong direction, which entails a renewed alignment and thus costs valuable time. This also leads to the fact that a target control is omitted during the treatment.

Although it is part of the prior art from German published patent application DE 10 2006 060 070 A1, with a lithotripsy device, to detect the position of the sound wave treatment device with respect to the patient support in spatially fixed coordinates by a complicated space navigation system, in order in this manner to also achieve an automatic positioning of the target point with regard to the focus of the sound wave treatment device, the usual manual control or post correction, which is regularly required with the treatment, is however effected in the same disadvantageous manner as that previously described.

### BRIEF SUMMARY OF THE INVENTION

Against this background, it is the object of the invention to design a medical therapy device, in particular of the previously described type, such that the operation is simplified and thus is more secure, in particular with regard to the positioning of the target point located in the patient body into the treatment center of the treatment device, thus for example into the focus of the sound wave treatment device.

According to the invention, this object is achieved by a display and operating unit according to the following description and the drawings.

The medical therapy device according to the invention comprises at least one picture-providing locating device, a treatment device and a patient support. It further comprises means for the motorized positioning at least of the treatment device relative to the patient support, means for the control of the therapy device, and a display and operating device, on which the treatment center of the treatment device may be represented in at least one picture of the locating device. According to the invention, operating elements are provided on the display and operating device, with which the positioning in coordinates of the locating device may be controlled.

According to the invention, a display and operating device, preferably in the form of a display and operating unit, is provided, which has a display, on which at least one picture of a locating device is represented or may be represented, in relation to a treatment center of a treatment device, and which further comprises operating elements, with which the treatment center may be displaced in the coordinates of the picture of the locating device, for the purpose of positioning the patient.

The solution according to the invention may basically be applied with the most varied of therapy devices, particularly advantageous however with therapy devices in the form of extracorporal sound wave treatment devices, wherein the treatment device is a focusing sound wave treatment device, and the treatment center is formed by the focus of the sound wave treatment device, thus with those therapy devices as are known from the initially mentioned prior art. Thereby, the positioning is typically effected by a suitable relative movement between the patient support and at least the treatment device, typically however the treatment device together with one or more locating devices. Basically, it is thereby of no importance whether the treatment device is moved to the patient support, or the patient support to the treatment device. Usually, however, the positioning is effected by way of a suitable displacement of the patient support.

The basic concept of the present invention is thus no longer to activate the positioning between the treatment device and the patient support, which is usually effected by displacing the patient support in table coordinates (up-down, right-left, and forwards-backwards), in these table coordinates, but in the picture coordinates of the locating device, thus in the coordinates of the display of the locating device or the locating devices, if several are envisaged. The treating physician may thus typically carry out the positioning according the coordinates of the locating device, which are visible on the display, by the operating elements, typically four or six operating elements for moving in two or three space coordinates, and thus does not need to waste any energy considering the conversion of display coordinates into table coordinates, but may position the patient in the shortest path in a targeted manner and in a manner such that the target point in the body, for example the concrement, is brought to agree with the focus of the sound wave treatment device. Here, it is insignificant whether the treatment device is moved relative to the patient or the patient is moved relative to the treatment device, but in practice it has proved to be effective to displace the patient support in a motorized manner. In order to move the focus of the sound wave treatment device in the direction of the target point in the body, thus to carry out the previously described positioning, the therapist merely needs to operate suitably marked operating elements, on which the movement direction is represented, as would be necessary on moving the focus on the displaced picture in the direction of the target point. If, for example, the target point lies in the right upper picture quadrant with respect to the focus in the displaced picture, then the operating elements are to be actuated, which point to the right and upwards with respect to the represented picture, until an agreement exists.

Thus, due to the design according to the invention, not only a significantly quicker positioning is effected than with the prior art, but also importantly in a significantly safer and more reliable manner in the desired direction, which is why the danger practically no longer exists that the concrement is lost from the picture of the display. The solution according to the invention, moreover, even during the treatment, constantly permits a control and correction, insofar as the target point and focus have dislocated, i.e. on account of the breathing movement of the patient.

Advantageously, an ultrasound locating device is provided as a locating device, whose picture is represented at least on the display of the display and operating device. It is to be understood that additionally, the picture of this locating device may be represented on a separate display, as is usual with the prior art.

According to a further embodiment of the invention, alternatively or advantageously additionally, an X-ray locating device, preferably an X-ray C-arm, whose picture is represented at least on the display of the display and operating device, is provided as a locating device. Here too, it is to be understood that a representation of the picture on a further screen is possible. Since in particular an X-ray C-arm may also be applied in other ways, insofar as this is concerned, it may be useful to provide the representation on the display of the display and operating device according to the invention, additionally next to the usual representation on a screen.

It is particularly advantageous with lithotripsy devices to provide an ultrasound locating device as well as an X-ray locating device. The pictures of the locating devices should usefully be represented next to one another, in each case simultaneously with a representation of the focus of the sound wave treatment device on the display of the display and operating device. Since the previously mentioned locating devices provide different information with regard to the spatial position as well as with regard to the representation, it makes particular sense to provide the pictures of both locating devices next to one another on the display, wherein then usefully suitable operating elements for positioning are assigned to each picture representation and correspond to the coordinates of the respective picture.

The display and operating device according to a further embodiment of the invention advantageously comprises at least one touch-screen for the display of the picture or the pictures of the locating device(s), and moreover contains at least the operating elements for positioning. Such a design is particularly advantageous, since the operating elements may be arranged close to the picture or in the picture, and the positioning results by manual activation of the operating elements in a quasi-intuitive manner and furthermore is visible in the current picture. Advantageously, not only the operating elements for positioning, but also all remaining operating elements of the therapy device are arranged in a clear manner on the touch-screen, since then the complete operation of the device including the location guidance and target guidance may be effected by way of the touch-screen.

Here, the operating elements are designed as key fields, which without further instruments, may advantageously only be activated by finger contact. Insofar as the operating elements for positioning are concerned, it is advantageous to design the respective key fields as arrow key fields and to arrange them at the lateral edges of the respective picture representation on the touch-screen. A positioning is then effected by touching the arrow keys, and specifically in a manner such that a target cross, representing the target focus of the sound wave treatment apparatus, is brought to correspond with the target in the picture of the locating device by touching the respective arrow keys. Here, the target cross typically remains in the middle of the locating picture, while the target moves over the picture by actuating the respective arrow keys, and thus the patient is positioned relative to the therapy apparatus and locating apparatus. Whereas with the picture of the X-ray locating device, advantageously four arrows are arranged at the picture edges for the two-dimensional displacement, at the picture edges of the ultrasound locating device, advantageously in each case a diagonally running arrow key is provided at two corner points, additionally to these four arrows, and these in each case permit a positioning in the depth direction, that is in the third dimension of the picture.

It is to be understood that the key arrangements are assigned to the respective picture coordinates, i.e., for example, an actuation of an operating element in the picture of the ultrasound locating device to the bottom effects an upward movement or downward movement of the patient table, whereas an actuation of the equally directed arrow on the picture of the X-ray locating device next to this effects a displacement of the table in a horizontal direction. This, however, may also be in the same direction depending on the arrangement of the locating devices.

A particularly practical handling results if the display and operating unit is designed as one unit, in particular by a preferably cable-less touch-screen. This permits an operation from each position of the therapist, as the case may be, even protected behind a wall which is shielded to the X-rays.

For positioning, it is particularly advantageous to design the patient support in a motorized displaceable manner in three space coordinates. A data conversion into picture coordinates, with regard to software, may then be effected in the control.

With the therapy device according to the invention, it is not only possible to provide a manual semi-automatic positioning of the patient relative to the treatment device, but according to a further embodiment of the invention, also an automatic positioning of the center of the treatment device, in particular of the focus of the sound wave treatment device, to a target previously determined on the display, thus an automatic mode for target guidance. For this, after activating e.g. the target guidance, the therapist first marks the target region in the patient on one of the two pictures of the locating devices with a marker (this may be a second target cross, a pen or the finger of the user), thus for example the concrement, whereupon the automatic mode is activated and the positioning is effected automatically. One may also effect an automatic target tracking during the treatment in this automatic mode.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. Shown in the drawings are:

FIG. 1 is a simplified schematic, perspective representation of a therapy device according to an embodiment of the invention;

FIG. 2 is representation of a design of a touch-screen layout according to an embodiment of the invention;

FIG. 3 is an alternative design of the touch-screen layout for the representation of the picture of an ultrasound locating device; and

FIG. 4 is a further view of the design of the touch-screen layout of FIG. 3 for the alternate representation of the picture of an X-ray locating device.

### DETAILED DESCRIPTION OF THE INVENTION

The therapy device represented by FIG. 1 is a lithotripsy device with a patient support 1, whose rest 2, on which the patient lies during the treatment, may be displaced electro-motorized in three space coordinates. A lithotripter 3 is envisaged as a treatment device, whose treatment head 4 producing focusing sound waves is pivotably arranged next to the rest 2 of the patient support 1, which is recessed in this region. Furthermore, one provides an X-ray locating device 5, whose X-ray picture intensifier 6 in the representation according to FIG. 1 is arranged at a distance above the rest 2. The X-ray locating device 5 is mechanically coupled to the treatment head 4 of the lithotripter 3 and may be pivoted about a horizontal axis in a manner known per se, in order to permit a three-dimensional locating. The picture of the X-ray locating device 5 may be represented on an X-ray screen 7. Moreover, an ultrasound locating apparatus 8 is provided, whose locating head 21 is arranged within the treatment head 4 and comprises an ultrasound screen 9 for representing the ultrasound picture.

The previously described parts 1 to 9 of the device, including the associated control electronics and operating devices, are known per se from U.S. patent application publication 2008/0146908 A1, and are therefore not described in detail here.

The ultrasound wave treatment device represented by FIG. 1 may be operated by a display and operating unit in the form of a touch-screen 10, which is connected to the control of the therapy device, in particular also the locating devices, via radio. The therapist 11 represented here by example, who holds the touch-screen 10, may by the touch-screen 10 control all functions of the device which are significant with regard the treatment, and may simultaneously see the pictures 12, 13 of both locating devices, specifically of the X-ray device 5 and of the ultrasound locating device 8.

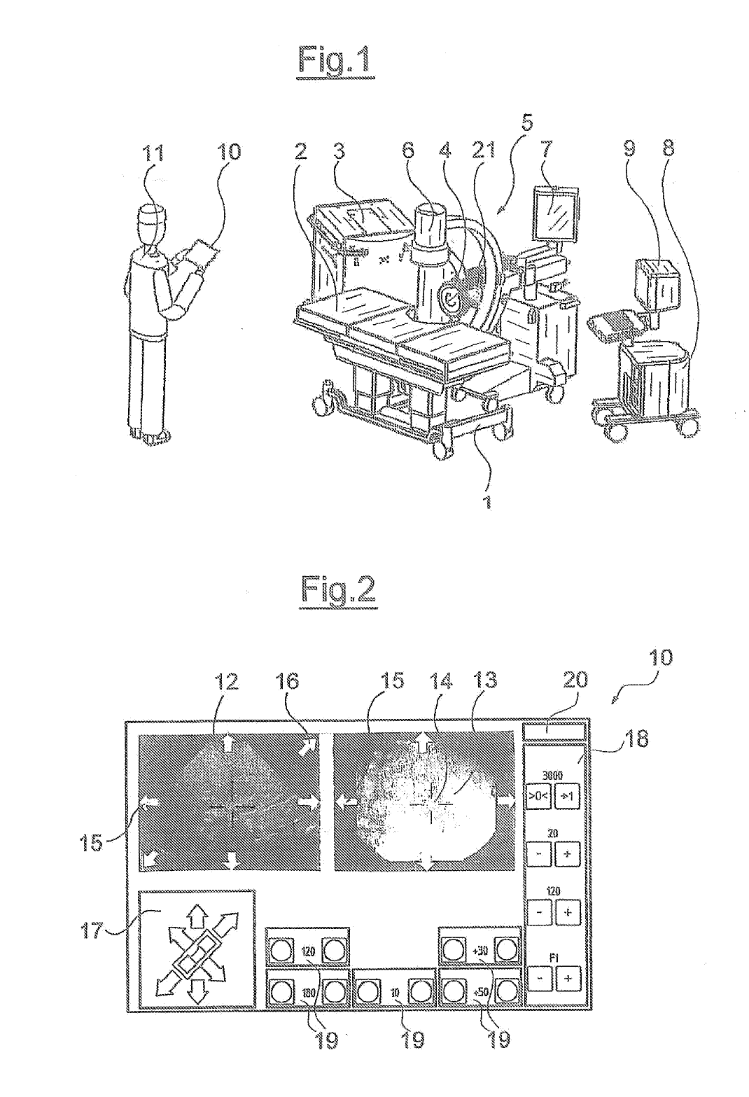

In FIG. 2 the picture 12 of the ultrasound locating device 8 is represented on the left next to the picture 13 of the X-ray locating device 5 on the touch-screen 10. The focus of the treatment head 4 is represented in the picture in the form of a target cross in both pictures 12, 13. In each case, four arrow keys 15 are provided at the edges of each of the pictures 12 and 13, and these are touch sensitive and on contact effect a positioning of the rest 2 of the patient support 1 with respect to the focus point of the treatment device 3, 4, which is represented by the target cross 14. Here, the control is such that the arrow keys 15 are aligned according to the picture coordinates of the pictures 12 and 13, so that the respective picture with the target point located therein is pushed quasi below the target cross 14, according to the directions of the respective arrow keys 15, whereby the patient rest 2 with the patient located thereon is positioned accordingly. This is effected until the therapist has reached an agreement between the target cross 14 and the target in the body of the patient.

Since the arrow keys 15 in both pictures 12 and 13 only permit a positioning in two dimensions, diagonally arranged arrow keys 16 are provided in the corners of the picture 12 of the ultrasound locating device 8, with which keys a positioning in the third dimension, thus in a direction perpendicular to the picture plane of the touch screen 10, may be effected.

The arrow keys 15 and 16 are designed in a manner known per se as touch-sensitive keys and the arrangement is effected by way of suitable software activation of the touch-screen 10. An operating field 17 is provided independently of the arrow keys 15 and 16, which permit a positioning of the patient on the basis of the locating pictures 12 and 13 relative to the focus 14, and the patient support may be displaced in table coordinates with this operating field, as is useful for a coarse alignment of the patient for making out the target region. The six arrow keys represented there are likewise designed as touch-sensitive operating fields and permit a positioning of the support 2 to the front, the rear, left, right, top and bottom.

Moreover, an operating field 18 is provided on the right edge of the touch-screen 10, which forms the shockwave operating field, with which the therapy head 4 may be activated for treatment. Further operating fields 19 for different functions of the device are likewise provided on the touch-screen 10, in order thus to ensure a uniform operation on a screen.

Apart from the previously described semi-automatic positioning of the patient or the rest 2 with respect to the focus of the therapy head 4, there is also the possibility of triggering an automatic positioning by activating an automatic mode. For this, first a key field 20 is to be activated, whereupon the target region is marked in one or both of the locating pictures 12 and 13 by way of a marker (second target cross, pen or the finger of the user) and is activated after actuating a trigger key. Then, after a suitable computation, an automatic positioning is effected automatically in a manner such that the location marked on the pictures 12 and 13 is brought to agree with the target cross 14, thus the focus of the therapy source.

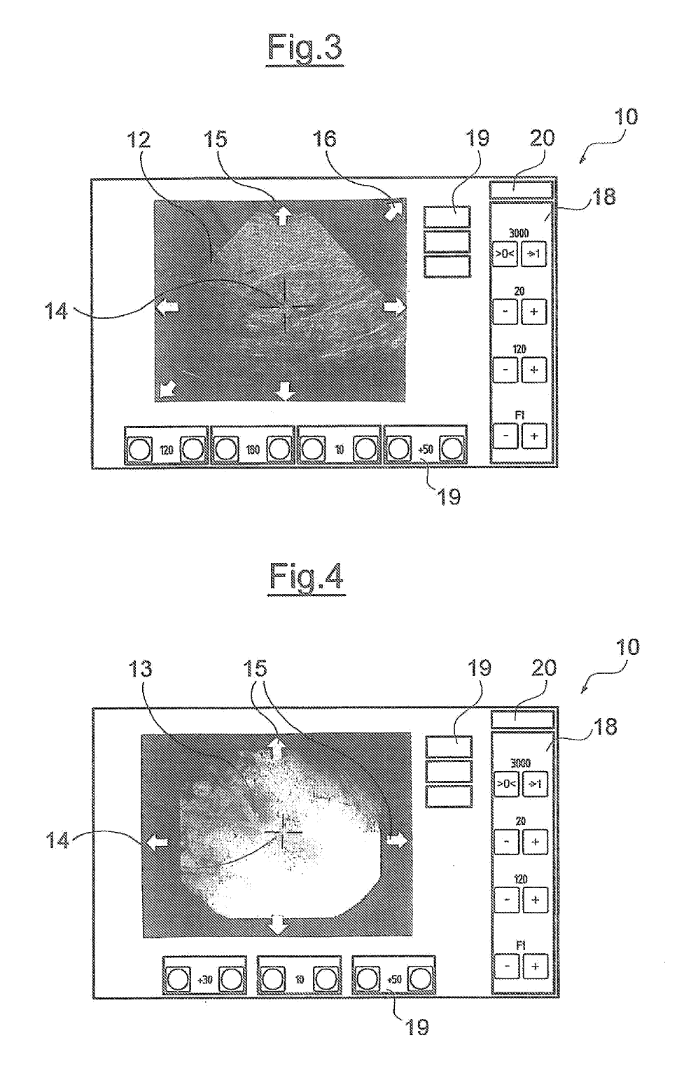

A touch-screen 10 is represented in FIGS. 3 and 4, which is assigned to a therapy device which selectively displays an ultrasound locating picture 12 (FIG. 3) or an X-ray locating picture 13 (FIG. 4). The positioning is effected in the same manner as previously described, but it may likewise be effected in auto-mode or in a semi-automatic manner by actuation of the arrow keys 15, 16 by the therapist 11. It is to be understood that also with the use of only one locating system, as is represented by FIGS. 3 and 4, further operating fields 17, 18, 19, as for example the operating field 17 for the control of the rest 2 in space coordinates, or other functions, may be integrated.

As the embodiment variants according to FIGS. 2 to 4 illustrate, by the touch-screen 10, not only may in-picture navigation be effected in the simplest manner, but moreover also one may envisage practically infinite operating functions. Here, the locating picture or pictures 12, 13 may be represented at a suitable location and in a suitable size on the screen, just as the operating fields 17 to 19. Also, as the case may be, a first representation on the screen 10 may serve exclusively for in-picture navigation, and a second representation effected with suitably assigned operating fields 17 and 19, whereby one merely needs to provide one switch-over function on the screen 10. The operating fields 17 to 19 may also be adapted to practically any therapy devices in a suitable manner. Different representation types/colors/groupings may be offered to the user, so that the therapist may set up the representation on the screen 10 in a quasi individual manner, according to his needs. The respective arrangements and representations may be implemented in a simple manner with regard to software, so that changes may also be carried out, if for example an apparatus of the therapy device is replaced by another.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-15. (canceled)

16. A medical therapy device comprising at least one locating device (5, 6; 8) providing a picture, a treatment device (3, 4), a patient rest, means for motorized positioning at least of the treatment device (4) relative to the patient rest (2), means for controlling the therapy device, and a display and operating device on which a treatment center (14) of the treatment device (3, 4) may be represented in at least one picture (12, 13) of the locating device (5, 6; 8) and on which operating elements (15, 16) are provided to control the positioning in coordinates of the locating device (5, 6; 8).

17. The therapy device according to claim 16, which is a lithotripsy device, wherein the treatment device is a focusing sound wave treatment device (3, 4), and the treatment center (14) is formed by a focus of the sound wave treatment device (3, 4).

18. The therapy device according to claim 17, wherein the locating device is an ultrasound locating device (8), whose picture (12) is represented at least on a display of the display and operating device.

19. The therapy device according to claim 18, further comprising an X-ray locating device (5, 6), whose picture (13) is represented at least on the display of the display and operating device.

20. The therapy device according to claim 19, wherein the X-ray locating device (5, 6) is an X-ray C-arm (5).

21. The therapy device according to claim 19, wherein the pictures (12, 13) of two locating devices (5, 6, 8) are represented next to one another, in each case with the focus of the sound wave treatment device (3, 4) simultaneously on the display of the display and operating device.

22. The therapy device according to claim 16, wherein the display and operating device comprises at least one touch-screen (10) for display of the at least one picture (12, 13) of the at least one locating device (5, 6, 8), the touch-screen containing at least the operating elements (15, 16) for positioning.

23. The therapy device according to claim 22, wherein the operating elements (15, 16) for positioning comprise key fields at lateral edges of the display of the at least one picture (12, 13) on the touch-screen (10).

24. The therapy device according to claim 23, wherein the key fields are arrow key fields.

25. The therapy device according to claim 22, wherein the display and operating device comprises a cable-less touch-screen.

26. The therapy device according to claim 16, wherein the patient rest (2) is displaceable in a motorized manner in three space coordinates.

27. The therapy device according to claim 16, comprising an automatic mode wherein an automatic positioning of the treatment center (14) of the treatment device (3, 4) is effected onto a target previously determined on a display.

28. The therapy device according to claim 17, comprising an automatic mode wherein an automatic positioning of the focus of the sound wave treatment device is effected onto a target previously determined on a display.

29. The therapy device according to claim 22, wherein further operating fields (17, 18, 19) for control of the therapy device are provided on the touch-screen (10).

30. A display and operating device for a medical therapy device, comprising a display on which at least one picture (12, 13) of a locating device (5, 8) is displayed in relation to a treatment center (14) of a treatment device (4), and operating elements (15, 16) for positioning represented on the display, wherein the treatment center (14) is moved in coordinates of the at least one picture (12, 13) of the locating device (5, 8) to position a patient receiving therapy.

31. The display and operating device according to claim 30, wherein the display comprises a touch-screen (10) containing at least the operating elements (15, 16) for positioning.

32. The display and operating device according to claim 31, wherein the operating elements for positioning comprise key fields (15, 16) at lateral edges of at least one picture (12, 13) of the locating device (5, 8) on the touch-screen (10).

33. The display and operating device according to claim 32, wherein the key fields (15, 16) are arrow key fields.

34. The display and operating device according to claim 31, further comprising operating fields (17, 18, 19) for control of the therapy device represented on the touch-screen (10).

35. A display and operating device for the medical therapy device according to claim 16, comprising a display on which at least one picture (12, 13) of a locating device (5, 8) is displayed in relation to a treatment center (14) of a treatment device (4), and operating elements (15, 16) for positioning represented on the display, wherein the treatment center (14) is moved in coordinates of the at least one picture (12, 13) of the locating device (5, 8) to position a patient receiving therapy.
